(19) United States
(12) Patent Application Publication
(10) Pub. No.: US 2020/0152304 A1
(43) Pub. Date: May 14, 2020

(54) Systems And Methods For Intelligent Voice-Based Journaling And Therapies

(71) Applicant: Kintsugi Mindful Wellness, Inc., San Francisco, CA (US)

(72) Inventors: Grace Chang, San Francisco, CA (US); Rima Seiilova-Olson, West Sacramento, CA (US)

(21) Appl. No.: 16/684,415

(22) Filed: Nov. 14, 2019

Related U.S. Application Data
(60) Provisional application No. 62/767,281, filed on Nov. 14, 2018.

(51) Int. Cl. G16H 20/00; G10L 25/63; G10L 15/26; G06F 17/27

(52) U.S. Cl. CPC G16H 20/00; G06F 17/2705; G10L 15/26; G10L 25/63

(57) ABSTRACT

A system for providing therapies from voice-based journaling, the system includes a processor, and a memory communicatively coupled to the processor. The memory itself includes a journaling logic to receive a plurality of voice journal entries from a user. The received voice journal entries include at least voice data and contextual data. An analyzer logic is also included to extract textual data from the plurality of voice journal entries, generate a sentiment analysis score based on the textual data, generate an emotional classification score based on the voice data, the textual data and the contextual data, and determine user recommendations as therapies based on the sentiment analysis score, emotional classification score, voice data, textual data, and contextual data. A user interface logic then displays the user recommendations to the user and can receive, from the user, feedback data associated with the user recommendation to be used in future user recommendations.

### PRIORITY

This application claims the benefit of priority to U.S. Provisional Application, No. 62/767281, filed Nov. 14, 2018, the entirety of which application is incorporated herein by reference.

### FIELD

Embodiments of the disclosure relate to the field of voice-based journaling. More specifically, certain embodiments of the disclosure relate to a system, apparatus and method for generating personalized and effective mental health therapies and recommendations through intelligent processing of data related to voice-journaling

### BACKGROUND

Mental health is often heavily underserved in America and to an even greater degree abroad. According to the Institute for Medicaid Innovation (IMI), it is increasingly recognized that “non-clinical factors contribute significantly to the health outcomes of society. Clinical care is only one factor responsible for ten to fifteen percent of preventable mortality in the United States. A range of other factors, collectively categorized as social determinants of health (SDOH), have a more profound influence on care, outcomes, and population health. The World Health Organization (WHO) has found that SDOH contributes to sixty percent of preventable mortality.

There is a corpus of studies which validate that the expression of emotionally upsetting experiences by writing or talking improves physical health (i.e., journaling), enhances immune function, and results in fewer visits to medical practitioners. Key research in expressive writing include surprising findings for various problems including asthma and rheumatoid arthritis with 20 percent improvement in lung function and a 28 percent reduction in disease severity, memory function for college freshman with better working memory after 7 weeks, wound healing on punch biopsy in the upper arm with significantly smaller wounds 14 days after the puncture, irritable bowel syndrome improved disease severity, blood pressure and infectious disease improvements for African-Americans and homosexual men, and improved employment outcomes for job seekers, just to name a few. It has been found that 84 percent of surveyed women both domestically and abroad have well-understood daily challenges; however, less than 2 percent had a mitigating workflow to address ongoing stress, anxiety, and depression.

Journaling therapies can be based on Pennebaker's Paradigm, which has shown that in over two hundred clinical trials, expressive writing helps patients with stress, anxiety, and depression immediately following treatment and months beyond. Talking about our problems and the frequency at which we articulate these concerns into a journal provides a rich historical account much more accurate than recounting how we feel at the doctor's office in ten to fifteen-minute appointments. This is important because historical records can reduce skew on how our days actually went. The current challenge of mental health assessments like the PHQ-9 and GAD-7 evaluations is that they suffer from recency bias.

Despite the understood benefits of journaling therapy, people may often find it difficult to access proper services. Beyond the oftentimes prohibitive costs, the number of available therapists may be insufficient to render proper care to the potential pool of patients. For example, as of 2010, Zimbabwe has eighteen mental health practitioners to serve the entire country, while China has only a mere 27,000 practitioners to support its 1.4 billion population.

### SUMMARY

[Systems and methods for providing therapies from voice-based journaling in accordance with embodiments of the invention are disclosed. In one embodiment, the system includes a processor, a memory communicatively coupled to the processor, the memory including a journaling logic to receive a plurality of voice journal entries from a user the received voice journal entries comprise at least voice data and contextual data, an analyzer logic configured to extract textual data from the plurality of voice journal entries, generate a sentiment analysis score based on the textual data, generate an emotional classification score based on the voice data, the textual data and the contextual data, and determine at least one user recommendation based on the sentiment analysis score, emotional classification score, voice data, textual data, and contextual data, and a user interface logic configured to display the at least one user recommendation to the user, and receive, from the user, feedback data associated with the at least one user recommendation.

In another embodiment, the contextual data includes journal entry metadata.

In further embodiments, the contextual data includes manual classification data.

In more embodiments, only the voice data is utilized to extract the textual data.

In a variety of embodiments, the at least one user recommendation is a therapy.

In a some embodiments, the therapy is a clinical therapy.

In various other embodiments, the therapy is a non-clinical therapy.

In still further embodiments, the system utilizes an external computing service to generate the sentiment analysis score.

In yet additional embodiments, the external computing service is an on-demand cloud-based computing service.

In further additional embodiments, the system utilizes an external computing service to generate the emotional classification score.

In yet further embodiments, the external computing service is an on-demand cloud-based computing service.

In still yet additional embodiments, the system further includes a communication logic configured to extract personal identification data from the voice data, the textual data, and the contextual data to generate non-identifying voice data, non-identifying textual data, and non-identifying contextual data, generate at least one anonymous identification marker the anonymous identification marker can be utilized to recognize the source of transmitted data, transmit the non-identifying voice data, non-identifying textual data, non-identifying contextual data, and at least one anonymous identification marker to the on-demand cloud-based computing service, receive an emotional classification score from the on-demand cloud-based computing service, and provide the emotional classification score to the analyzer logic for determination of the at least one user recommendation.

In many embodiments, a method to provide therapies from voice-based journaling, includes receiving a plurality of voice journal entries from a user the voice journal entries comprise at least voice data, and contextual data, extracting textual data from the plurality of voice journal entries, generating a sentiment analysis score based on the textual data, generating an emotional classification score based on the voice data, the textual data and the contextual data, determining at least one user recommendation based on the sentiment analysis score, emotional classification score, voice data, textual data, and contextual data, providing the determined at least one user feedback to the user, and receiving from the user, feedback data associated with the at least one user recommendation.

In various embodiments, the method further includes displaying, on a computing device display, at least one determined visual feedback response the visual feedback response determination is based on at least the sentiment analysis score, emotional classification score, voice data, textual data, and contextual data.

In some embodiments, the journal entry metadata includes global positioning system (GPS) data.

In more embodiments, the journal entry metadata includes time and date data.

In a variety of embodiments, the received feedback data is utilized to determine subsequent user recommendations.

In still more embodiments, the method utilizes an external computing service to generate the emotional classification score.

In more further embodiments, the external computing service is an on-demand cloud-based computing service.

In a number of embodiments, a system for providing therapies from voice-based journaling, includes a processor, a memory communicatively coupled to the processor, the memory including a journaling logic to receive a plurality of voice journal entries from a user the received voice journal entries comprise at least voice data and contextual data, an analyzer logic configured to extract textual data from the plurality of voice journal entries, generate a sentiment analysis score based on the textual data, generate an emotional classification score based on the voice data, the textual data and the contextual data, determine at least one user recommendation based on the sentiment analysis score, emotional classification score, voice data, textual data, and contextual data, and determine at least one visual feedback response the visual feedback response the visual feedback response is based on at least the sentiment analysis score, emotional classification score, voice data, textual data, and contextual data, and a user interface logic configured to display the at least one user recommendation to the user, display the at least one visual feedback response to the user, and receive, from the user, feedback data associated with the at least one user recommendation.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 depicts a system diagram of the voice-based journaling and therapy system in accordance with various embodiments of the invention.

FIG. 2A depicts an abstract illustration of the components of a voice-based journaling and therapy computing device in accordance with various embodiments of the invention.

FIG. 2B depicts an abstract illustration of journal entry data in accordance with an embodiment of the invention.

FIG. 3 depicts an exemplary diagram of a voice-based journaling and therapy computing device in communication with various cloud-based services in accordance with various embodiments of the invention.

FIG. 4 is an exemplary flowchart of a voice-based journaling and therapy process in accordance with various embodiments of the invention.

FIG. 5 is an exemplary flowchart of a cloud-based voice-based journaling and therapy process in accordance with various embodiments of the invention.

FIG. 6 is an exemplary flowchart of a crowdsourced voice-based journaling and therapy process in accordance with various embodiments of the invention.

### DETAILED DESCRIPTION

The following description is not to be taken in a limiting sense but is made merely for the purpose of describing the general principles of exemplary embodiments. The scope of the disclosure should be determined with reference to the claims. Reference throughout this specification to “one embodiment,” “an embodiment,” or similar language means that a particular feature, structure, or characteristic that is described in connection with the referenced embodiment is included in at least the referenced embodiment. Likewise, reference throughout this specification to “some embodiments” or similar language means that particular features, structures, or characteristics that are described in connection with the referenced embodiments are included in at least the referenced embodiments. Thus, appearances of the phrases “in one embodiment,” “in an embodiment,” “in some embodiments,” and similar language throughout this specification can, but do not necessarily, all refer to the same embodiment.

Further, the described features, structures, or characteristics of the present disclosure can be combined in any suitable manner in one or more embodiments. In the description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure.

In the following description, certain terminology is used to describe features of the invention. For example, in certain situations, the term “logic” is representative of hardware, firmware and/or software that is configured to perform one or more functions. As hardware, logic (or engine) may include circuitry having data processing or storage functionality. Examples of such circuitry may include, but are not limited or restricted to a microprocessor, one or more processor cores, a programmable gate array, a microcontroller, a controller, an application specific integrated circuit, wireless receiver, transmitter and/or transceiver circuitry, semiconductor memory, or combinatorial logic.

Logic may be software in the form of one or more software modules, such as executable code in the form of an executable application, an application programming interface (API), a subroutine, a function, a procedure, an applet, a servlet, a routine, source code, object code, a shared library/dynamic link library, or one or more instructions. These software modules may be stored in any type of a suitable non-transitory storage medium, or transitory storage medium (e.g., electrical, optical, acoustical or other form of propagated signals such as carrier waves, infrared signals, or digital signals). Examples of non-transitory storage mediums may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the executable code is stored in persistent storage.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

In response to the problems discussed above, embodiments of the present disclosure provide for intelligent voice-based journaling and therapy systems to provide users with access to mental health care benefits from their personal computing devices. Embodiments herein describe a system for utilizing neural-network and other machine learning models configured for recording and analyzing voice journal entries. Embodiments of the system can analyze the raw text and audio from natural conversation to discover speech patterns indicative of depression or other mental health conditions. In response to determined mental states, the system can provide user recommendations that may include clinical and non-clinical therapies based on past user preferences and feedback. User recommendations may also be suggested based on data derived from feedback received from other users within the system. To avoid bias, the system may allow users to edit and correct emotions and dedicated topics. The more information gets contextualized to a user, the better the system algorithms can be in avoiding biases.

More broadly, the systems described herein can provide an on-demand, smart platform for users to talk and vent on their challenges or share their gratitude journal entries with others like them. Expressive writing or pathophysiology of disclosure are well-documented solutions for alleviating stress as shown in Pennebaker's expressive writing paradigm, but there isn't an easy way to do this regularly, nor is there a feedback loop from which individuals can learn. Advancements in machine learning methods, along with the pervasiveness of sensors and smart devices, and consumer demand for voice-enabled products have made voice journaling possible today. By using various machine learning models, embodiments of the system can infer a user's emotional state and facilitate otherwise difficult behavioral workflows. Beyond recognizing emotional triggers, users can gain valuable personal insight by viewing and learning about how their identified voice biomarkers can be associated with various mental states, and/or recent levels of sleep, nutrition, outdoor activities, or other physiological events. By adjusting personal habits in response to these insights, users can work to track to positive outcomes and generate positive feedback.

Machine learning models for analyzing voice-based journal entries can utilize datasets using natural language tools, and detect the emotions expressed in the journal entry. In certain embodiments, emotions can be categorized into Ekman's 6 basic emotions (Joy, Anger, Disgust, Fear, Sadness, Surprise) and Shame. If no particular emotion is detected, the model can classify the emotion as “none”. Further machine learning models can be trained on a dataset to extract the person (e.g., family, friend, kids, manager, partner, peer, kids, pets, other, none) and the context (home, work, school, in-transit, other, none) discussed. It would be understood by those skilled in the art that embodiments of the invention can utilize other emotions or categorizations beyond those explicitly outlined here.

Another core functionality is on deep customizations and personalized recommendations. As the system learns more about users, more relevant recommendations can be generated immediately following creating a journal entry. Providing contextual recommendations when a user is sad, anxious, or stuck in a negative loop can ensure a healthy ongoing dialogue and care for the users' wellbeing. Often, events in life trigger users to focus on mental health. For example, a breakup in a relationship, a move, a new baby, and/or switching jobs may greatly affect the types of mental states and recommendations that will be relevant and effective for the user. As more data is gathered from the user, as well as the global user population, more relevant and successful customized recommendations may be provided for these different scenarios.

The voice-based journaling and therapy system can have multiple overall system architectures. A first architecture may include serving contextually relevant community journal entries to satisfy the user's need to feel validated. Another architecture can be to serve contextually relevant recommendations to help the user derive the source of stress, anxiety, and depression with further questions like cognitive and/or dialectical behavioral therapy exercises and to temporarily soothe distress with mindfulness exercises and preferred relaxation exercises. The community generated by the voice-based journaling and therapy system can help the user feel validated. Journal entries can help the user feel heard and understood. Recommendations may help the user solve the source of the problem or allay any immediate pain. Goals help the user plan or remind herself of what is effective or key insights to remember

User tests have demonstrated that users respond positively to visual feedback. Thus, visual feedback may be incorporated into the voice-based journaling system to help produce positive outcomes. For example, a visual feedback system may utilize Deepmoji which feeds text through a 3-layer bi-long short-term memory model with attention and returns the most appropriate emoji. Visual feedback will be provided to make the voice-based journaling experience more engaging and fun.

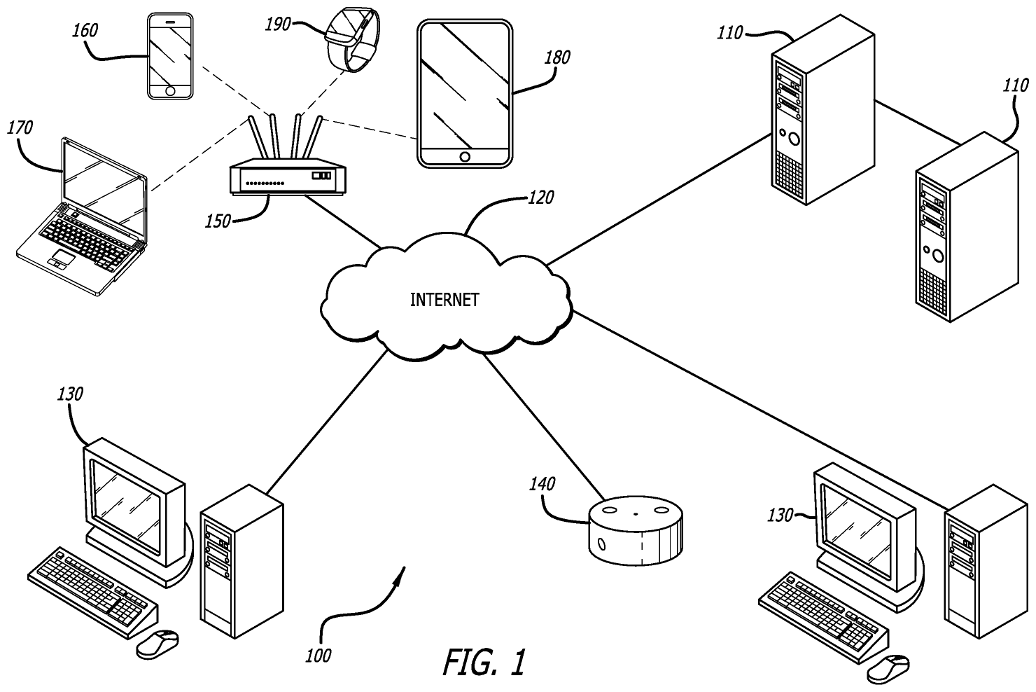

Referring to FIG. 1, a system diagram of the voice-based journaling and therapy system 100 in accordance with an embodiment of the invention is shown. The voice-based journaling and therapy system 100 comprises a plurality of devices that are configured to transmit and receive data related to providing, recording, and processing voice-based journal entries to generate a plurality of customized and responsive therapies. In many embodiments, voice-based journaling servers 110 are connected to a network 120 such as, for example, the Internet. Voice-based journaling servers 110 are configured to transmit a variety of data across the network 120 to any number of computing devices such as, but not limited to, personal computers 130, personal listening computing devices 140, and mobile computing devices including laptop computers 170, cellular phones 160, portable tablet computers 180 and wearable computing devices 190. In additional embodiments, voice-based journaling and therapy data may be mirrored in additional cloud-based service provider servers or edge network systems. In still additional embodiments, the voice-based journaling servers 110 can be hosted as virtual servers within a cloud-based service.

In further embodiments, the sending and receiving of voice-based journaling and therapy system data can occur over the network 120 through wired and/or wireless connections. In the embodiment depicted in FIG. 1, the mobile computing devices 160, 170, 180, 190 are connected wirelessly to the network 120 via a wireless network access point 150. It should be understood by those skilled in the art that the types of wired and/or wireless connections between devices on the voice-based journaling and therapy system 100 can be comprised of any combination of devices and connections as needed.

In various embodiments, the voice-based journaling and therapy system 100 may broadly accept voice-based journal entry from users via any number of personal computers 130, personal listening computing devices 140, and/or mobile computing devices 160, 170, 180, 190. These voice-based journal entries may, in some embodiments, generate a plurality of data related to the journal entry, and determine a customized therapy as a response to the content of the journal entry data. The customized response may be generated from a list of pre-determined responses within the personal computers 130, personal listening computing devices 140, and/or mobile computing devices 160, 170, 180, 190. In other embodiments, the customized therapy may be received from the voice-based journaling servers 110.

In still further embodiments, the journal data may be stripped of personal identifying data and transmitted to the voice-based journaling servers 110 and/or other cloud-based services for processing. The processed data is then returned to the personal computers 130, personal listening computing devices 140, and/or mobile computing devices 160, 170, 180, 190 for output to the user. Based on the therapy suggested, the user may subsequently generate feedback data that can be transmitted back to the voice-based journaling servers 110 that may utilize the feedback data to further improve the modeling of various machine learning algorithms that can then be utilized to generate better therapy suggestions for future users.

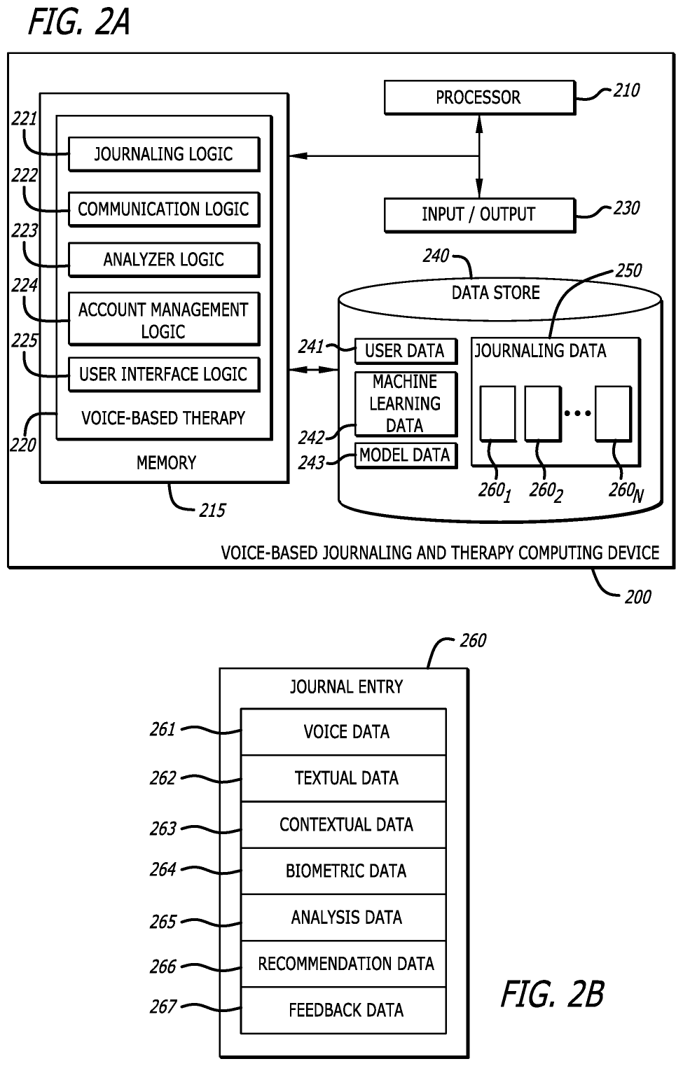

Referring to FIG. 2A, an abstract illustration of the components of a voice-based journaling and therapy computing device 200 in accordance with various embodiments of the invention is shown. A voice-based journaling and therapy computing device 200 may be any computing device that can accomplish a voice-journaling and therapy process. These computing devices may include personal computers 130, personal listening computing devices 140, and/or mobile computing devices 160, 170, 180, 190 as described in FIG. 1 or may comprise any computing device sufficient to receive, transmit, and respond to voice-based journal entries from users. In many embodiments the voice-based journaling and therapy computing device 200 can comprise at least a processor 210, memory 215, inputs and outputs 230, and a data store 240. The memory 215 can include a voice-based therapy application 220 that may further comprise journaling logic 221, communication logic 222, analyzer logic 223, account management logic 224, and user interface logic 225. The data store 240 may include user data 241, machine learning data 242, model data 243, and journaling data 250 which may further comprise a plurality of journaling entry data 2601, 2602, 260N.

In a variety of embodiments, journaling logic 221 can be configured to facilitate the acquisition of journal entries from one or more users. Journal entries typically require voice data to be acquired from a user providing a voice-based journal entry. Journaling logic 221 may work with the user interface logic 225 to provide a user with one or more tools and/or prompts to facilitate recording of a voice-based journal entry. Journaling logic 221 may utilize one or more recording codecs or algorithms to store the voice data provided by the user. Some embodiments may use provided methods and/or toolkits from the operating system of the voice-based journaling and therapy computing device 200.

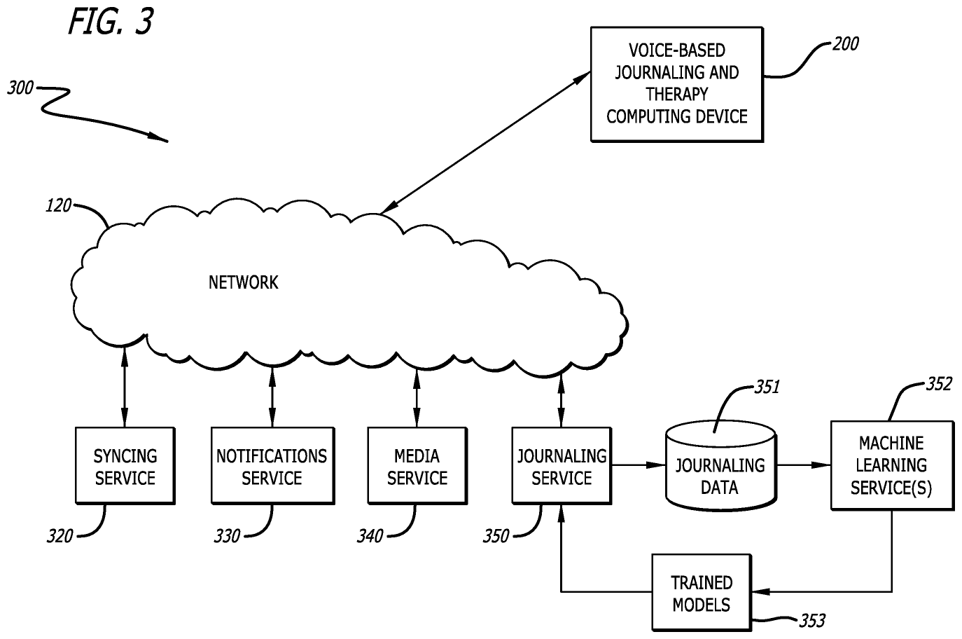

In additional embodiments, communication logic 222 can facilitate transfer of data between the voice-based journaling and therapy computing device 200 and external services. The descriptions of the external services are described in more detail below in the discussion of FIG. 3. In some embodiments, the communication logic 222 can establish communication channels with the external services via network connections. Certain embodiments may utilize network connection tools provided by the operating system of the voice-based journaling and therapy computing device 200.

In further embodiments, analyzer logic 223 facilitates the processing and generation of supplemental data related to journal entries. Journal entries typically begin with the recording of voice data 261. Many embodiments of the analyzer logic 223 can take the voice data 261 and process it to generate textural data 262, which subsequently be used to generate analysis data 265 such as a sentiment score. Analyzer data 223 may also process the textual data 262 along with other available data sources to generate emotional classifications and/or topic classifications. In further embodiments, the analyzer logic 223 can also generate one or more user recommendations based on all available journal entry data 260.

The analyzer logic 223 may also utilize user data 241, machine learning data 242 and/or model data 243 to support the processing and generation of the journal entry data 260. In various embodiments, analyzer logic 223 may utilize external services to facilitate the processing and/or generation of journal entry data 260. In other embodiments, the analyzer logic 223 may download user data 241, machine learning data 242, and/or model data 243 from an external source that can then be utilized to process and/or generate journal entry data 260.

In more embodiments, account management logic 224 can provide methods for users to log in, establish, and update their personal account information. Users may be prompted to log in and log out of their account for security reasons. The voice-based journaling and therapy system may benefit from understanding data related to the user. In various embodiments, account management logic 224 may facilitate the input of personal data from a user. For example, the user may be prompted to enter basic biographical info (birthdate, location, etc.) but may also be prompted to enter additional such as mental health and/or medical history. Finally, the account management logic may work with the user interface logic 225 and analyzer logic 223 to provide feedback data associated with the user.

In many embodiments, user interface logic 225 can generate one or more graphical or audio user interfaces to provide methods for users to navigate through the voice-based journaling and therapy process. The user interface logic 225 may provide prompts to the user to enter journal entries, user account data, and/or feedback on previous user recommendations.

In some embodiments, user data 241 relates to any data associated with a specific user. This can be identifying data such as name, birthdate, location, core values, relaxation preferences, contacts, etc. User data may also comprise historical trends, and/or habits relating to voice-based journaling including dates of previous journal entries.

In various embodiments, machine learning data 242 can be utilized by the analyzer logic 223 to process journal entry data 260 to generate supplemental data that can be stored as analysis data 265. Machine learning data can comprise specific algorithms related to generating sentiment scores, topic classifications, emotional classifications, and/or clinical diagnoses.

In still more embodiments, model data 243 may include full data models to be utilized by the analyzer logic 223 in order to generate supplemental data for the journal entry data 260. Data stored within the model data 243 may also comprise a plurality of weights that may be utilized within a neural network or other similar device.

In further embodiments, journaling data 250 comprises a plurality of journal entry data 2601-260N which may be configured such that each journal data entry 260 includes data relating to a specific journal entry. This may be done to keep certain data siloed away from other entries. More detailed discussion of the structure of journal entry data 260 is below with respect to FIG. 2B.

Referring to FIG. 2B, an abstract illustration of journal entry data 260 in accordance with an embodiment of the invention is shown. As described above with reference to FIG. 2A, journal entry data 260 can exist within a journal data store 250 and can be unique to each journal entry that is captured by a user. The journal entry data 260 in FIGS. 2A-2B is depicted as being portioned and stored based on the individual journal entries associated with the data. Further discussion of the types of data that can be found within journal entry data 260 is below.

In many embodiments, voice data 261 comprises the raw audio data that is captured with a microphone or other recording device during the voice-based journaling process. This voice data 261 can comprise waveform data and can be formatted into any audio format desired based on the application and/or computing resources. For example, limited storage resources may lead to using increased compression algorithms to reduce size, while computing resources may limit the amount of compression that can be done on the fly. Voice data 261 can be stored in lossy or lossless formats. In some embodiments, the voice data may be processed before storage or utilization elsewhere within the voice-based journaling and therapy system. Pre-processing can include noise-reduction, frequency equalizing, normalizing, and or compression. Such pre-processing may increase the amount of supplemental data that can be generated from the voice data 261.

In additional embodiments, textual data 262 comprises text representing the words that were spoken within the captured voice data 261. The voice-based journaling and therapy system may utilize any available automated transcription methods or algorithms to extract the words spoken therein. In certain embodiments, the textual data 262 may be generated by an external service that can more efficiently produce the textual data 262. Textual data may often be stored merely as a text-based file for future reference.

In further embodiments, contextual data 263 comprises any supplemental data that can be generated and associated with the captured voice data 261. In some embodiments, contextual data 263 may comprise relevant account data, journal entry metadata, manual classification data, and/or voice marker data. Relevant account data may be any data associated with the user that may be utilized to gain insight into the captured voice data 261. For example, user data may indicate that a user has their birthday and may then be utilized to further gain understanding (or at least generate an additional data point) when processing their voice data 261 and other subsequent data. Journal metadata can include any additional data relating to the event of capturing the journal entry. Some journal metadata examples may include the global positioning system (GPS) coordinates of where the journal entry was taken (on vacation, at work, etc.), the time at which it was taken (late at night or first thing in the morning, etc.), what was the quality of the recording, and/or how long the recording was. Manual classification data can include any data that the user generated or entered relating to classifying or describing the journal entry data 260. By way of example and not limitation, the user may be prompted to rate their feelings prior to the journal entry or may label their emotional responses afterward.

Voice marker data can include any data derived from analyzing the voice data 261 for stressors, inflections, tone, or any other vocal characteristic that may be indicative of mental state. For example, the voice-based journaling and therapy system may attempt to analyze the voice data 261 to find pitch variances or stress when pronouncing specific words, phrases, and or syllables. Unique aspects of voice biomarkers can facilitate the identification of depression and anxiety through voice journal entries and measure how recommendations affect trends in severity of depression and anxiety, or for neither, baseline “healthy” mix of moods and emotions. They may also help the user understand emotional triggers and specifically what workflows influence an increase/decrease in depression or anxiety.

In certain embodiments, biometric data 264 can comprise any data generated or received from a wearable computing device 190 like the smartwatch depicted in FIG. 1. Wearable computing devices 190 may be a smartwatch, but may also be a heart-rate monitor, blood-pressure cuff, pedometer, exercise equipment, and/or other external health data available from the computing device operating system. Biometric data 264 can be utilized to further understand and adjust sentiments and emotions underlying the journal entry data 260. For example, correlations can be made (e.g., users are happier when they have gone for a run in the previous time block) or better internal understanding may occur (e.g., a specific user typically labels herself as mad when the journaling takes place late at night versus in the middle of the day.).

In a variety of embodiments, analysis data 265 can include various data generated as a result of machine learning processing of the journal entry data 260 by analyzer logic 223 (FIG. 2A). In some embodiments, the analysis data 265 includes a sentiment score based on the generated textural data 262. Furthermore, the analysis data may include emotional classification data. In certain embodiments, emotional classification data may be a plurality of emotional tags that can be associated with the journal entry data 260. Finally, in some embodiments, analysis data 265 may include topic classification data which may comprise an overarching topic related to the voice data 261, which may subsequently be used for recommendation generation by the analyzer logic 223.

In some embodiments, sentiment analysis may be an integer number (e.g., −1.0 to 1.0). Emotion classification can encompass a plurality of emotional states (e.g., joy, sadness, anger, fear, surprise, disgust, shame, etc.). Topic classifications may include general topics of journal entries extracted from textual data (e.g., family, work, money, health, relationship, etc.). In some embodiments, further cognitive distortion identifications may be generated (e.g., catastrophizing, overgeneralization, black and white thinking, etc.). The combination of sentiment analysis, emotion classification, topic classification, audio signal analysis, sleep, and fitness information can be used to make relevant suggestions to the user in some embodiments.

In some embodiments, recommendation data 266 comprises various recommendations generated by the voice-based journaling and therapy computing device 200 in response to the journal entry data 260. User recommendation data 266 may include data related specifically to the promoted recommendation but may also be utilized to store secondary recommendations that may be provided as alternatives to the main recommendation.

Recommendations may take the form of identifying a certain overall emotion, the action urge of that emotion, and recommending the opposite action. For example, a sad emotion, may prompt a user to stay in bed or to be alone. The voice-based journaling and therapy system may generate recommendations in response to various emotions similar to the examples below:

| Emotion | Action Urge | Opposite Action for Recommendation |
|---|---|---|
| Sad | Be alone, stay in bed | Be around others, get active |
| Angry | Yell, attack, | Be extra kind, no judgements, |
| | be judgmental | gently avoid |
| Frustrated | Give up | Try even harder |
| Betrayed | Hurt or revenge | Forgiveness |
| Worthless | Harm self | Help others |
| Fear | Run away, avoid | Stay and do what is fearful |
| Guilt | Repair transgression | Do what makes you feel |
| | | guilty and ashamed |
| Shame | Hide | Be public |

In certain embodiments, recommendation data 267 may further comprise at least one visual feedback response to the user generated by the analyzer logic 223. Visual feedback data may comprise emojis, videos, and/or other picture data that can be presented to a user after a voice-based journal entry has been completed.

In more embodiments, feedback data 267 comprises any data that is subsequently generated and/or received in relation to previously suggested user recommendations. In many embodiments, the feedback data 267 comprises a user's response to the generated user recommendation and/or visual feedback.

Those skilled in the art will recognize that the journal entry data discussed herein with respect to FIG. 2B is only a single representation of potential journal data. For example, various embodiments may have journal entry data 260 pooled together such that all voice data 261 is stored together, all textual data 262 for all journal entries is stored together, etc. Furthermore, other methods of storing journal entry data 260 may exist wherein certain aspects may be stored externally while other aspects are stored locally. One illustrative example would be journal entry data 260 that stores voice data 261, and feedback data 267 externally, but stores textual data 262, contextual data 263, biometric data 264, analysis data 265, and/or recommendation data 266 locally to avoid exposing private data.

Referring to FIG. 3, an exemplary diagram of a voice-based journaling and therapy computing device in communication with various external computing services 300 in accordance with various embodiments of the invention is shown. In a variety of embodiments, the generation of therapies in response to voice-based journaling can be accomplished entirely within a single voice-based journaling and therapy computing device 200. However, in some embodiments, external computation power is desired and/or required. For example, a personal listening computing device 140 (FIG. 1) may have the computational power to receive voice data from journal entries but may not have the computational power to utilize machine learning methods to parse the voice data, generate sentiment scores, and/or emotional classifications. In other embodiments, the voice-based journaling and therapy computing device 200 may be within a public or semi-public setting which may provide security/privacy issues with having data stored directly onto the machine. In either of these example cases, the use of external computing services 300 can be beneficial.

All external computing services may be reached through communication over a network 120 such as, but not limited to, the Internet. In some embodiments, a syncing service 320 can attempt to provide consistent user experiences over multiple voice-based journaling and therapy computing devices. As an illustrative example, a user may initially set up their voice-based journaling account on a personal computer 130 (FIG. 1) and subsequently provide a voice-based journal entry by talking with a personal listening computing device 140, and finally add further journal entries away from home on their cellular phone 160. A syncing service 320 may attempt to keep user data similar across all devices such that the user experience is not changed between devices. This can happen by, for example, promulgating all user account data and changes across all connected devices, providing past journal entries at any computing device, and/or updating processes, training models, and/or general software updates between various devices within the same account.

In certain embodiments, a notifications service 330 can provide external notifications via push or pull methods. These notification services may be in lieu of, or in addition too, notification services that are part of the various operating systems of the voice-based journaling and therapy computing devices 200. Notifications can be prompts to keep using the service, in hopes of creating a habit or to provide insight into past collected data (e.g., providing a notification that certain behaviors may be beneficial and/or detrimental in response to detecting the user within a specific area via GPS signals, etc.).

In further embodiments, a media service 340 may provide hosting or delivery of supplemental media for the voice-based journaling and therapy computing device 200. In certain embodiments, media may be provided to the user as a training video, calming and/or meditation session, or an affirmation video as a customized user recommendation.

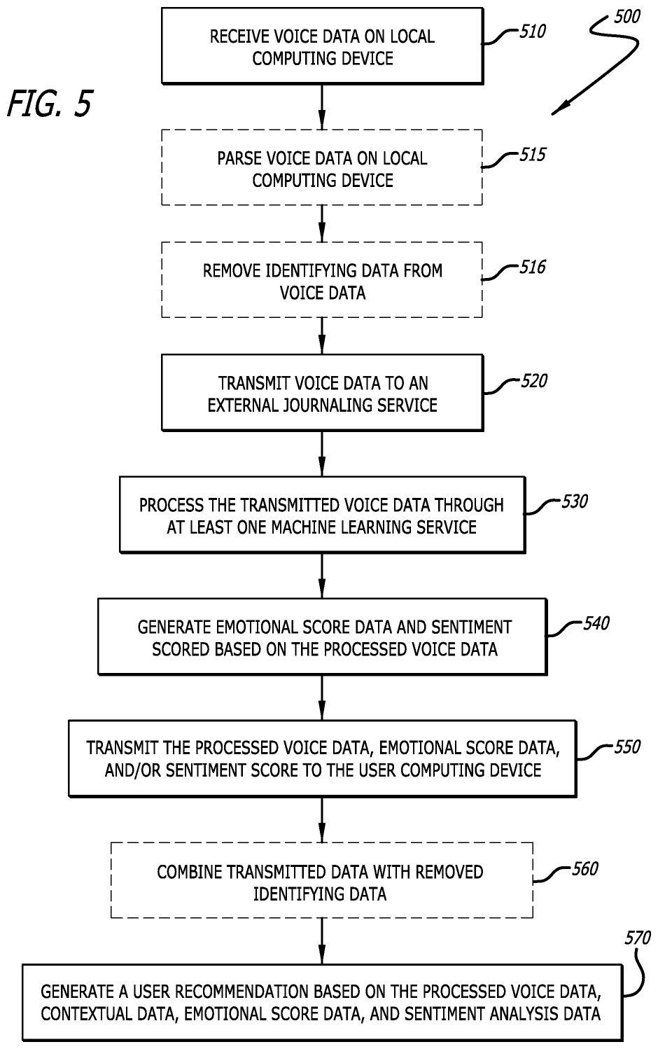

In many embodiments, a journaling service 350 may accept, process, and return various journal entries. Many of the processes that can occur within a voice-based journaling and therapy computing device 200 may also be offloaded to the journaling service 350. The received data can be stored within a journaling data store 351 for processing. In many embodiments, processing of the journaling data store 351 occurs within one or more machine learning services 352. The exact methods and types of machine learning methods utilized by the journaling service 350 are discussed in more detail above. Many of the machine learning services 352 however, utilize at least one or more trained models 353 to help generate the desired from within the journal entry data. As discussed below with respect for FIG. 5, the presence of various external computing services 300 can be understood to comprise various embodiments wherein the type and/or amount of data processed externally over a network 120 can vary in amount based on the application desired.

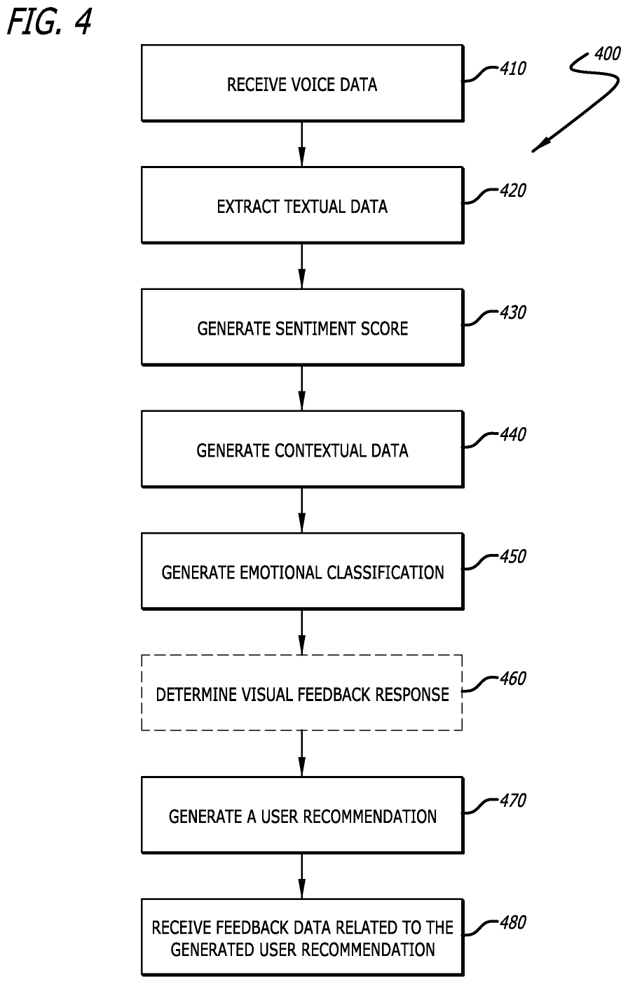

Referring to FIG. 4, an exemplary flowchart of a voice-based journaling and therapy process 400 in accordance with various embodiments of the invention is shown. The process 400 begins with the receiving of voice data (block 410). In many embodiments, the voice data is received through a voice-based journaling and therapy computing device 200 (FIG. 2) as part of a voice journal entry by a user. The received voice data is often in a raw waveform format. The voice data can then be processed to extract textual data (block 420). In some embodiments, textual data comprises text recognized from the voice data. The process 400 may subsequently utilize the textual data to generate a sentiment score (block 430). In certain embodiments, the sentiment score can be a number between negative one and positive one associated with a negative or positive sentiment.

Contextual data may be generated from data from other data sources within the process 400 (block 440). For example, contextual data may comprise relevant account data, journal entry metadata, manual classification data, and/or voice marker data associated with the voice data. Utilizing all available data sources, the process 400 may generate an emotional classification that may label at least one portion of the journal entry with an emotional classification (block 450). In many embodiments, the generation of the emotional classification is done via at least one machine learning method. With the various data sources received and/or generated, the process 400 may optionally determine a visual feedback response to provide the user (block 460). Additionally, the process 400 can generate a user recommendation based on the available received and/or generated data sources (block 470). User recommendations may, in some embodiments, be generated by utilizing one or more machine learning methods. Generated user recommendations can be displayed on a user's voice-based journaling and therapy computing device and are typically therapeutic in nature. At some subsequent time, the process 400 may conclude by receiving feedback data related to the generated user recommendation (block 480). In a variety of embodiments, feedback data may be manually entered by a user (e.g., by responding to a prompt for user ratings, etc.) or automatically generated (receiving usage and/or external data that corresponds to user recommendations such as calling someone or working out for example).

Referring to FIG. 5, an exemplary flowchart of a cloud-based voice-based journaling and therapy process 500 in accordance with various embodiments of the invention is shown. Similar to the process 400 outlined in FIG. 4, the process 500 herein utilizes at least one external computing service to generate user recommendations. The process 500 can begin with voice data being received on a local computing device such as a voice-based journaling and therapy computing device (block 510). This is typically accomplished by recording one or more voice-based journal entries. The recorded audio can be stored as voice data for further processing. Depending on the application desired, the process 500 may optionally parse some of the voice data on the local computing device (block 515). In some embodiments, parsing may include generating contextual data from the received voice data. In other embodiments, the parsing may be accomplished by an external computing device, such as a voiced-based journaling server.

Before transmission, the process 500 can optionally remove identifying data from the voice data (block 516). In some embodiments, the identifying data may include metadata or ownership information (e.g., associated account name, source internet protocol (IP) address, etc.) while other embodiments may extract waveform data that is recognized to comprise sensitive and/or personally identifying information. Often, this step is utilized to comply with various data and medical privacy standards and/or laws.

Once prepared the process 500 may transmit the voice data to an external journaling service (block 520). As discussed above, journaling services may comprise one or more external computing machines that can receive and process journal entry data. In embodiments where at least some of the voice data has been parsed on the local computing device, textual data may also be transmitted along with the voice data. The external journaling service may further process the transmitted voice data through at least one machine learning service (block 530). Often, the machine learning service attempts to extract various data from the voice data as well as generate supplemental data associated with the transmitted voice data. In embodiments where textual data was transmitted from the local computing device, the machine learning may generate a sentiment score based on the received textual data. In more embodiments, the machine learning service may be utilized to generate emotional score data based on the processed voice data (block 540). Additionally, in embodiments where no textual data was sent from the local computing device, the process 500 can generate textual data from the received voice data and utilize the generated textual data to further generate a sentiment score.

Once generated on the external journaling service, the data can then be sent back to the local computing device for further processing, if necessary (block 550). This data may include any of voice data, emotional score data, and/or sentiment scores. Once received back at the local computing device, the process 500 can, in embodiments where identifying data was removed prior to transmission to the external journaling service, restore the removed identifying data to the data received back from the external journaling service (block 560). Once the data has been restored, the process 500 may then generate a user recommendation based on the processed, generated, and received data (block 570).

Those skilled in the art will recognize that utilization of external services within the process 500 may include more or fewer steps within the user recommendation generation process. In some embodiments, all of the data within the process may be received, processed, and/or generated within an external computing device. External computing devices may be physical hardware servers remotely located, cloud-based servers that can be utilized on command, or virtual servers within an on-demand processing service.

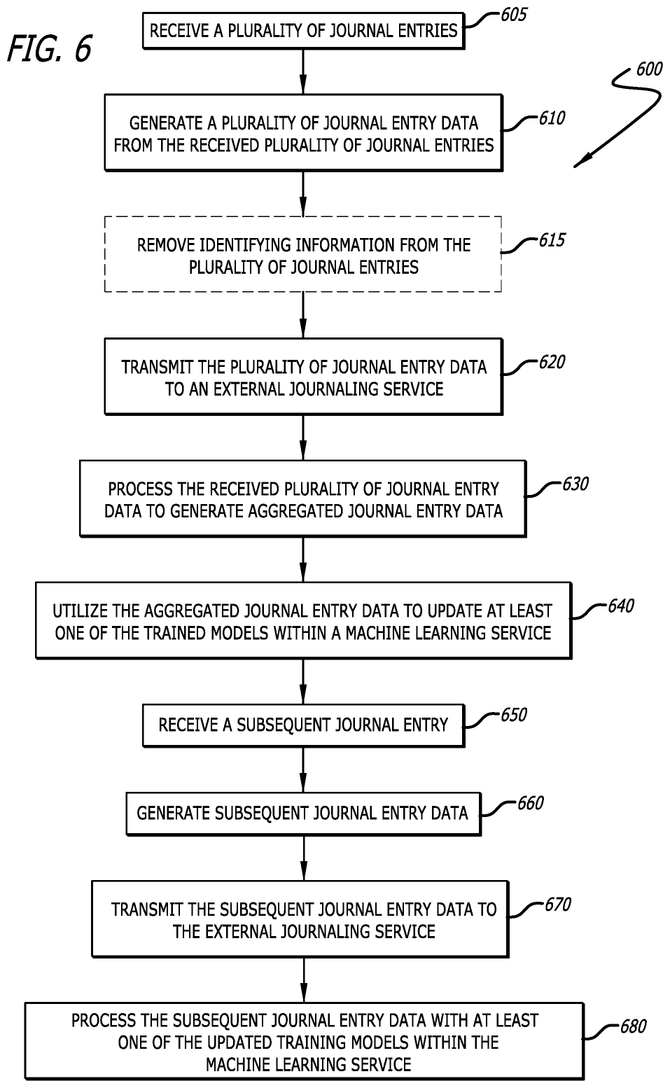

Referring to FIG. 6, an exemplary flowchart of a crowdsourced voice-based journaling and therapy process 600 in accordance with various embodiments of the invention is shown. As discussed above, embodiments of the invention herein can utilize data extracted from various user's journal entry data to better train machine learning models which can more accurately identify data within captured voice data and may offer more user-approved recommendations based on past individual and/or group feedback. To accomplish this, various embodiments of the process 600 can first receive a plurality of journal entries that can span any number of users or any amount of time (block 605). The various journal entries can be utilized to generate a plurality of journal entry data such as those depicted in FIG. 2B (block 610).

In many embodiments, before transmission to an external source, the process 600 can optionally remove identifying information from the plurality of journal entries (block 615). As discussed above with respect to FIG. 5, the process 600 can comply with various data and medical privacy laws during transmission of data. The transmission of journal entry data can be made to an external journaling service (block 620). Often, the external journaling service exists remotely and can only be communicated with over the Internet. In response to receiving the journal entry data, processing can be done to generate aggregated journal entry data (block 630). As those skilled in the art can recognize, aggregated journal entry data may include more than just a sum of all journal entry data received. Processing may be done to determine patterns, matches, and/or trends within the various journal entry data. One method of utilizing the aggregated journal entry data is to update at least one of the machine learning models (block 640). These machine learning models may be within an external machine learning service or, in some embodiments, may be personalized model within a local computing device.

Subsequent to updating one or more machine learning models, the process 600 can receive additional journal entries (block 650). These new journal entries may be from the same user or from a new user. As a result of receiving new journal entries, more journal entry data is generated (block 660). The newly generated journal entry data can then be transmitted to an external journaling service for processing (block 670). Once received, the external journaling service may process the newly generated journal entry data with at least one of the updated machine learning training models (block 680). In certain embodiments, the external journaling service has access to a machine learning service. In other embodiments, the external journaling service may have to further transmit the newly generated journal entry data to another external machine learning service.

In the foregoing description, the invention is described with reference to specific embodiments thereof. It will, however, be evident that various modifications and changes may be made thereto without departing from the broader spirit and scope of the invention as set forth in the appended claims.

What is claimed is:

1. A system for providing therapies from voice-based journaling, the system comprising:
a processor;
a memory communicatively coupled to the processor, the memory comprising:
a journaling logic to receive a plurality of voice journal entries from a user wherein the received voice journal entries comprise at least voice data and contextual data;
an analyzer logic configured to:
extract textual data from the plurality of voice journal entries;
generate a sentiment analysis score based on the textual data;
generate an emotional classification score based on the voice data, the textual data and the contextual data; and
determine at least one user recommendation based on the sentiment analysis score, emotional classification score, voice data, textual data, and contextual data; and
a user interface logic configured to:
display the at least one user recommendation to the user; and
receive, from the user, feedback data associated with the at least one user recommendation.

2. The system of claim 1, wherein the contextual data comprises journal entry metadata.

3. The system of claim 1, wherein the contextual data includes manual classification data.

4. The system of claim 1, wherein only the voice data is utilized to extract the textual data.

5. The system of claim 1, wherein the at least one user recommendation is a therapy.

6. The system of claim 1, wherein the system utilizes an external computing service to generate the sentiment analysis score.

7. The system of claim 6, wherein the external computing service is an on-demand cloud-based computing service.

8. The system of claim 1, wherein the system utilizes an external computing service to generate the emotional classification score.

9. The system of claim 8, wherein the external computing service is an on-demand cloud-based computing service.

10. The system of claim 9, wherein the system further comprises a communication logic configured to:
extract personal identification data from the voice data, the textual data, and the contextual data to generate non-identifying voice data, non-identifying textual data, and non-identifying contextual data;
generate at least one anonymous identification marker wherein the anonymous identification marker can be utilized to recognize the source of transmitted data;
transmit the non-identifying voice data, non-identifying textual data, non-identifying contextual data, and at least one anonymous identification marker to the on-demand cloud-based computing service;
receive an emotional classification score from the on-demand cloud-based computing service; and
provide the emotional classification score to the analyzer logic for determination of the at least one user recommendation.

11. A method to provide therapies from voice-based journaling, the method comprising:
receiving a plurality of voice journal entries from a user wherein the voice journal entries comprise at least voice data, and contextual data;
extracting textual data from the plurality of voice journal entries;
generating a sentiment analysis score based on the textual data;
generating an emotional classification score based on the voice data, the textual data and the contextual data;
determining at least one user recommendation based on the sentiment analysis score, emotional classification score, voice data, textual data, and contextual data;
providing the determined at least one user feedback to the user; and
receiving from the user, feedback data associated with the at least one user recommendation.

12. The method of claim 11, wherein the contextual data comprises journal entry metadata.

13. The method of claim 11, wherein the contextual data includes manual classification data.

14. The method of claim 11, wherein the at least one user recommendation is a therapy.

15. The method of claim 11, wherein the method further comprises displaying, on a computing device display, at least one determined visual feedback response wherein the visual feedback response determination is based on at least the sentiment analysis score, emotional classification score, voice data, textual data, and contextual data.

16. The method of claim 11, wherein the journal entry metadata comprises global positioning system (GPS) data.

17. The method of claim 11, wherein the journal entry metadata comprises time and date data.

18. The method of claim 11, wherein the received feedback data is utilized to determine subsequent user recommendations.

19. The method of claim 11, wherein the method utilizes an external computing service to generate the emotional classification score.

20. The method of claim 19, wherein the external computing service is an on-demand cloud-based computing service.

21. A system for providing therapies from voice-based journaling, the system comprising:
a processor;
a memory communicatively coupled to the processor, the memory comprising:
a journaling logic to receive a plurality of voice journal entries from a user wherein the received voice journal entries comprise at least voice data and contextual data;
an analyzer logic configured to:
extract textual data from the plurality of voice journal entries;
generate a sentiment analysis score based on the textual data;
generate an emotional classification score based on the voice data, the textual data and the contextual data;
determine at least one user recommendation based on the sentiment analysis score, emotional classification score, voice data, textual data, and contextual data; and
determine at least one visual feedback response wherein the visual feedback response wherein the visual feedback response is based on at least the sentiment analysis score, emotional classification score, voice data, textual data, and contextual data; and
a user interface logic configured to:
display the at least one user recommendation to the user;
display the at least one visual feedback response to the user; and
receive, from the user, feedback data associated with the at least one user recommendation.
